(19) United States
(12) Patent Application Publication
(10) Pub. No.: US 2025/0099152 A1
(43) Pub. Date: Mar. 27, 2025

(54) ABLATION INSTRUMENT

(71) Applicant: Erbe Elektromedizin GmbH, Tuebingen (DE)

(72) Inventors: Marcus ADLER, Burladingen (DE); Felix KARCHER, Tuebingen (DE); Hanna ANDEL, Moessingen (DE)

(21) Appl. No.: 18/882,855

(22) Filed: Sep. 12, 2024

(30) Foreign Application Priority Data
Sep. 21, 2023 (EP) 23198816.3

(51) Int. Cl. A61B 18/02; A61B 18/00

(52) U.S. Cl. CPC A61B 18/02; A61B 2018/00083; A61B 2018/00101; A61B 2018/00178; A61B 2018/00577; A61B 2018/0262

(57) ABSTRACT

An ablation instrument having a fiber-reinforced thermoplastic plastic pin as well as a seat for s fluid line and a connection surface for the gas junction. The ablation instrument is particularly appropriate as single-use instrument as it is primarily made of plastic.

This application claims priority to European Patent Application No. 23198816.3, filed Sep. 21, 2023, the entirety of which is incorporated herein by reference.

The invention refers to an ablation instrument and a treatment device comprising such an ablation instrument. Thereby the invention particularly relates to CO₂-cooled thermo-ablation instruments that devitalize biological, living tissue by heat or cold treatment (cryotherapy). For heat creation in the tissue, electrodes can be provided on the instrument that introduce electrical current into the tissue, but are thereby cooled from the inside in order to keep the electrode tissue contact moist and electrically conductive. The invention, however, can also be used with instruments without electrodes influencing the tissue by cold treatment.

A cryoprobe is known from CN 1480103 A that comprises at its distal end an instrument tip cooled by means of the Joule-Thomson-effect. The instrument comprises an outer hose in the lumen of which a fluid line is arranged that supplies fluid to the instrument tip. The hose conveys back the fluid supplied to the instrument tip in the opposite direction. The hose is held in a handle at its (proximal) end located away from the patient. A sleeve consisting of fiber-reinforced plastic serves for this purpose, which is characterized by a low thermal conductivity.

Also, DE 10 2015 205 367 A1 uses thermal insulation characteristics of a fiber-reinforced plastic in a so-called cryo-tip.

Instruments of the initially mentioned type are, for example, also known from EP 2 630 982 B1. This instrument is provided with a plug at its proximal end having one or more pins extending in parallel away from a plug housing. They are configured to provide the instrument with cooling fluid. A cable having an electrical plug serving for the electrical supply of the instrument extends away from the plug.

It is the object of the invention to provide an instrument that can be operated with CO₂ and that allows simple manufacturing and thereby increase reliability and longevity.

This object is solved by the ablation instrument as well as particularly also by the treatment device incorporating the ablation instrument.

As mentioned, the ablation instrument according to the invention can be an instrument influencing tissue by treatment of heat or cold. In both cases, it comprises a hose surrounding a lumen, wherein the hose is configured in a closed manner at its distal end and is connected at its proximal end to a plug. This plug serves at least for supply of the ablation instrument with liquid, gaseous or also supercritical carbon dioxide (CO₂), for which purpose a fluid line originating from the plug extends through the lumen of the hose.

The plug comprises at least one pin connectable to the CO₂-source, having an inner channel that can be fluidically connected with the fluid line and that is closed at its outer end, located away from the plug. Two ring grooves on the pin arranged with distance to one another hold seals, particularly ring-shaped seals, for example O-rings. The seals can be inserted into the ring grooves as separate components or can be connected to the pin in a substance-bond manner. For this purpose, they can be produced together with the pin in a co-extrusion method. Between the two O-rings, a radial opening is formed leading into the channel.

According to the invention, the pin consists of a fiber-reinforced thermoplastic plastic, wherein particularly polycarbonate (PC), polyamide (PA), polybutylene polybutyterephthalate (PBT) or also polyimide (PI) can be considered as plastic. These plastics are preferably reinforced by short fibers embedded in the plastic and preferably regularly distributed therein. The fiber percentage can be between 20% and 40% by weight and is preferably 30% by weight (wt %). Particularly glass fibers can be considered as fibers, wherein the fiber length is preferably less than 3 mm, further preferably than 2 mm or also less than 1 mm. Other preferably non-metallic fiber materials that are inert against CO₂ and that in addition do not absorb CO₂ can also be used. Also, fibers of organic material, preferably, however, fibers from inorganic material can be used. The thickness of the fibers is preferably at approximately 5 μm to 10 μm and is preferably approximately 10 μm.

Fiber-reinforced plastics are able to withstand a long-term influence (longer than 60 minutes) of CO₂ being under high pressure (more than 50 bar), while the same plastics without fiber reinforcement show a significant strain after a certain influence duration. Such a strain can result in an elongation of the pin and indeed particularly in the area of its ring groove(s). Due to this elongation, a bad fit of the plug on a respective fluid socket may result. This is particularly the case if in the bushing an element contacting the face of the pin is provided, for example, a valve or a switch or simply an immovable abutment surface. In addition, in case of an elongation of the pin, a pin fracture or crack cannot be excluded. The invention remedies this and allows the use of the plug and therewith the instrument throughout an operation of multiple hours. For example, the ablation instrument, according to the invention, can be used for tumor ablation for which it is penetrated into a tumor. Electrodes provided on the instrument serve for current supply to the tumor and the cauterization thereof by means of heat buildup. In order to maintain the electrical contact between the tumor tissue and the electrodes that is necessary for this purpose, the electrodes are cooled from the inside. For this purpose serves the carbon dioxide conveyed under high pressure through the plug, which now withstands the carbon dioxide in spite of the configuration of its pins of plastic.

Electrical connectors that can be provided on the plug serve for current supply to the electrodes optionally provided on the ablation instrument. For example, such a connector can be formed by one or more contacts, which can be provided on one or more pins of the plug. In a preferred embodiment, the connector is formed by an electrical line having an electrical plug. The line can be a cable that extends from the plug, which establishes the fluid connection to the instrument, to another plug having electrical contact pins that can be inserted into an electrical generator provided for this purpose.

Preferably, the plug comprises a plug housing in which a plug unit is arranged. The plug unit is preferably configured as monolithic injection molded part that comprises a head arranged in a plug housing and at least one or multiple pins extending away therefrom parallel to one another. If only one pin is present, it can be configured in a stepped manner in order to connect multiple fluid channels. The pin unit is formed together with the pins monolithically without seam from the same fiber-reinforced thermoplastic plastic. It unifies together with a gas junction all necessary functional elements in order to connect the hose and the fluid line in a fluid-tight manner and to guide potentially present electrical lines out of the lumen of the hose in a gas-tight manner. In addition, it redirects the coaxial gas conveyance of the instrument to the parallel gas conveyance of the pins of the plug.

The material selection according to the invention allows to provide a particularly small wall thickness, particularly in the area of the grooves of the respective pin. This wall thickness can be smaller than the width of the respective groove measured in pin longitudinal direction. On the other side, it is facilitated to define a channel diameter being larger than the wall thickness in the area of the groove. This avoids undesired throttle effects in the gas path during the gas supply to the instrument and thus a good cooling effect at the distal end thereof.

Additional details of advantageous embodiments of the invention are subject matter of the figures, the drawing as well as the respective description or of claims. The drawing shows:

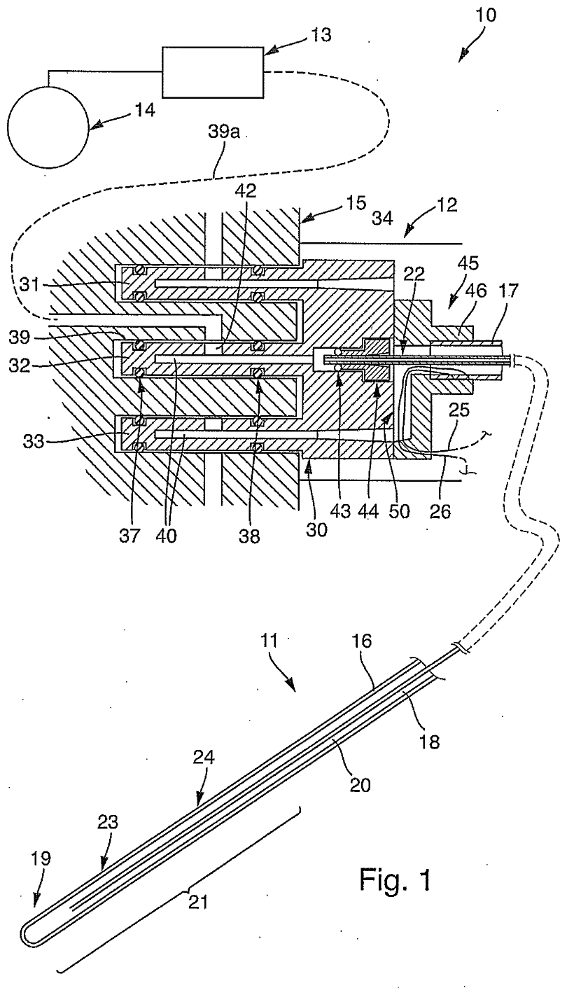

FIG. 1 a treatment device having an ablation instrument, the plug of which is connected to a socket of a supply apparatus that is connected with a CO₂ gas source,

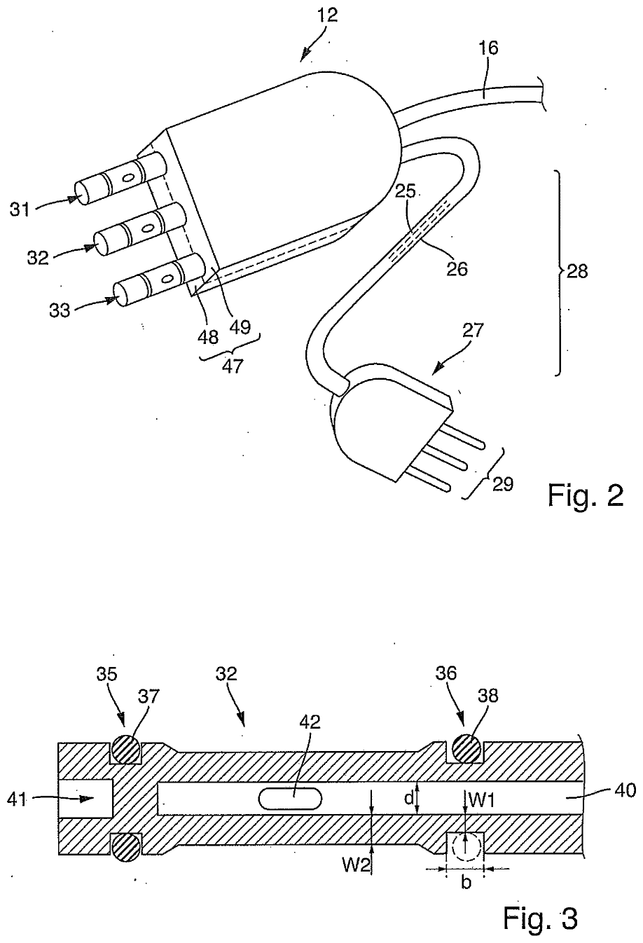

FIG. 2 the plug of the ablation instrument according to FIG. 1 in separate perspective illustration and

FIG. 3 a pin of the plug according to FIGS. 1 and 2 in a longitudinally cut and enlarged illustration in part.

In FIG. 1 a treatment device 10 is illustrated that comprises an ablation instrument 11 having a plug 12 provided at its proximal end. Also, part of the treatment device is a gas supply unit 13 that conveys carbon dioxide gas (CO₂) coming from a storage 14 to a gas socket 15.

The ablation instrument is preferably configured as single-use instrument that is not provided for multiple use on different patients and thus for reconditioning. Basically, however, it can also be designed as reusable instrument.

The ablation instrument 11 comprises a flexible hose 16 that can be introduced into a patient via a working channel of an endoscope, for example, wherein the proximal end 17 of hose 16 ends in the plug 12 and is held in a gas-tight manner. The hose encloses a lumen 18 that extends from the proximal end 17 up to the distal end 19 where the hose 16 and thus lumen 18 is closed.

Inside lumen 18 a fluid line 20 is arranged that serves for supplying CO₂ to the distal end section 21 of instrument 11 and hose 16. The proximal end 22 of fluid line 20 is in turn held in the plug 12 in a fluid-tight manner. The fluid line 20 can comprise one or more exit openings at its end in order to allow CO₂ to exit there that expands and absorbs heat, that means it cools.

In the distal end section 21 one or more electrodes 23, 24 can be arranged that serve supplying current and thus heating biological tissue. They can be connected via respective electrical lines 25, 26 with a plug 27 with which they form an electrical connector 28. The plug 27 can comprise one, two or more electrical contact pins 29 that serve supplying the electrodes 23, 24 via the lines 25, 26 with current. In addition, one or more of the contact pins 29 can be configured and used for instrument identification.

The plug 12 of the instrument 11 according to the invention comprises a pin unit 30 having at least one, however, preferably multiple pins 31, 32, 33 that extend away from a headpiece 34 parallel to one another with which they are seamlessly monolithically configured.

The following description of pin 32 in relation to FIGS. 1 and 3 applies accordingly for the other pins 31, 33. The pin 32 comprises a longitudinal tube-like base body on the outer side of which two ring grooves 35, 36 are arranged in axial distance to one another. The ring grooves 35, 36 serve to locate sealing elements therein, for example O-rings 37, 38 with which pin 32 is positioned in sealed manner in a corresponding jack 39 of gas socket 15. The pin 32 surrounds a channel 40, which is closed at the end 41 located away from plug 12. A radial opening 42 connects channel 40 with the interior of jack 39, if plug 12 is inserted into gas socket 15. The jack 39 is connected with the gas supply unit 13 via a fluid channel 39a and is via the latter supplied with pressurized (for example between 40 and 70 bar) CO₂.

The pin 32 has a wall thickness w1 at the ring groove 36 surrounding channel 40, which is smaller than the wall thickness w2 that is to be measured apart therefrom between ring grooves 35, 36. In addition, wall thickness w1 is preferably smaller than the groove width b as well as also smaller than channel diameter d.

The channel 40 opens out in a passage of headpiece 34, the passage also forming a seat 43 for holding the proximal end 22 of fluid line 20 in sealed manner. The fluid line 20 is held there against seat 43 in a sealed manner. At the proximal end 22 of fluid line 20, a holding piece 44 is secured by an adhesive.

On the headpiece 34, in addition, a gas junction 45 is held that is arranged in gas-tight manner on the side away from pins 31 to 33, for example a planar surface of headpiece 34, forming a mounting area 50. This mounting area 50 forms a seat for the gas junction. The gas junction serves for guiding the lines 25, 26 out of lumen 18, the redirection of the expanded CO₂ flowing backward to the pin, and concurrently secures holding piece 44 inside seat 43. The holding piece 44 and the gas junction 45 can thereby be configured monolithically. The hose 17 is secured in an opening of gas junction 45 by an adhesive 46. The gas junction 45 comprises a channel via which lumen 18 is connected to a channel 40′ of pin 33. In addition, gas junction 45 comprises a sealing or a sealing adhesive joint for the lines 25, 26, which serve for contacting electrodes 23, 24, in order to guide them out of the gas-conveying area of plug 12.

The plug 12 comprises a plug housing 47 that can consist, for example, of two housing shelves 48, 49 made of plastic, as indicated in FIG. 2. The headpiece 34 and gas junction 45 are mounted in the plug housing 47. The pins 31, 32, 33 project out of housing 47.

The treatment device 10 described so far operates as follows when using ablation instrument 11:

For devitalization of biological tissue, ablation instrument 11 is penetrated with its distal end section 21 into the tissue to be devitalized, for example a compact lung tumor, so that both electrodes 23, 24 are located inside the tumor. When activating instrument 11, gas supply unit 13 conveys carbon dioxide, taken from storage 14, under a controlled pressure in the range of 40 bar to 70 bar to the gas socket 15 and there particularly to the center jack 39. The respective gas conveying channel 39a thereby ends inside the jack between the two O-rings 37, 38 of the pin 32 inserted into jack 39.

Through the radial opening 42 and the channel 40, carbon dioxide flows now toward seat 43 and from there through fluid line 20 up to the distal end section 21 of the instrument in order to cool it and therewith also the electrodes 23, 24. The CO₂ passes there out of fluid line 20 into lumen 18 and expands thereby under remarkable cold development.

Concurrently, electrodes 23, 24 are supplied with electrical current via plug 27 or another connector 28, the current flowing through and heating the tumor thereby. The cooling of electrodes 23, 24 thereby avoids too excessive heating so that the maximum local temperature is not provided on the electrodes 23, 24, but in some distance thereto. This treatment process can require a remarkable time phase of, for example, 15 minutes. Also, the treating person can combine multiple of such treatments with one another in that he treats the respective tumor multiple times or treats (ablates) additional tumors accordingly. This means, that for a remarkable total duration, which can exceed an hour remarkably, carbon dioxide flows under a pressure of approximately 40 bar to 50 bar or more through the channel 40 of pin 32. Thereby, carbon dioxide diffuses into the plastic from which pin 32 is made, which can reduce the tensile strength of the plastic. Due to the fibers, particularly glass fibers, present in the plastic, however, a softening of the plastic and particularly an expansion in the area of the ring groove 36 is effectively inhibited. As a result, pin 32 maintains its length; it neither expands in pin longitudinal direction (direction of channel 40), neither the risk of fracture in the area of its small wall thicknesses (for example in the area of the ring groove 36) exists.

With the concept according to the invention, a plug 12 for an ablation instrument 11 is provided, which is—apart from potentially provided electrical lines 25, 26—entirely made of plastic and which can be produced in rational manner by economic manufacturing methods, for example by an injection molding technique. Particularly, the pin unit 30 contributes thereto, on which the pin 31, 32, 33 as well as seat 43 for fluid line 20 and a connection surface for the gas junction 45, consisting of plastic as well, are formed. In this configuration ablation instrument 11 is particularly appropriate as single-use instrument, the deposition of which does not require to dispose or recycle valuable materials, but only rationally producible plastic parts.

1. An ablation instrument, comprising:
a hose surrounding a lumen, wherein the hose extends longitudinally from a distal end section configured in a closed manner to a proximal end section coupled to a plug,
a fluid line extending through the lumen from the plug to the distal end section,
wherein the plug comprises at least one fiber-reinforced thermoplastic plastic pin configured to be connected to a gas supply and able to convey CO₂, the plug further having an interior channel connected to the fluid line,
wherein the at least one pin comprises two ring grooves circumferentially arranged with distance to one another and with sealing rings arranged therein between which a radial opening is formed leading into the interior channel.

2. The ablation instrument according to claim 1, wherein at least one electrode is arranged on the hose at the distal end section thereof, wherein the at least one electrode is connected to an electrical connector provided on the plug.

3. The ablation instrument according to claim 1, wherein the plug comprises a plug housing in which a pin unit is arranged.

4. The ablation instrument according to claim 1, wherein multiple pins are arranged on the pin unit extending parallel to one another that are seamlessly monolithically made of the same fiber-reinforced thermoplastic plastic.

5. The ablation instrument according to claim 1, wherein the channel arranged inside the pin is closed at its free end.

6. The ablation instrument according to claim 1, wherein the pin has a wall thickness (w1) in the area of the groove that is smaller than the wall thickness (w2) outside the groove.

7. The ablation instrument according to claim 6, wherein the pin has a wall thickness (w1) in the area of the groove that is smaller than half of the wall thickness (w2) outside the groove.

8. The ablation instrument according to claim 1, wherein the pin has a wall thickness (w1) in the area of the groove that is smaller than the width (b) of the groove measured in the pin longitudinal direction.

9. The ablation instrument according to claim 3, wherein the pin unit comprises a seat for mounting the fluid line in sealed manner.

10. The ablation instrument according to claim 3, wherein the pin unit comprises a mounting area for mounting a gas junction element that comprises a seat for mounting the proximal end of hose in sealed manner.

11. The ablation instrument according to claim 1, wherein the fiber-reinforced thermoplastic plastic is polycarbonate, polyamide polycarbonate, polybutylene, or polybutyterephthalate.

12. The ablation instrument according to claim 1, wherein the fiber-reinforced thermoplastic plastic comprises a fiber percentage of 20% by weight to 40% by weight.

13. The ablation instrument according to claim 1, wherein the fiber-reinforced thermoplastic plastic includes glass fibers.

14. The ablation instrument according to claim 1, wherein the fiber-reinforced thermoplastic plastic includes fibers having a length of less than 3 mm.

15. A treatment instrument comprising an ablation instrument in accordance with claim 1, and further comprising a CO₂ gas supply unit having a socket adapted to the plug of the ablation instrument, wherein the socket is connected to the CO₂ gas supply unit via a fluid channel.
